(19) United States
(12) Patent Application Publication
(10) Pub. No.: US 2023/0392196 A1
(43) Pub. Date: Dec. 7, 2023

(54) RNA retrieval process for preparing formalin-fixed, paraffin-embedded (FFPE) tissue samples for in situ hybridization

(71) Applicant: Applied Materials, Inc., Santa Clara, CA (US)

(72) Inventor: Erica, Mei Ling Teo, Singapore (SG)

(73) Assignee: Applied Materials, Inc., Santa Clara, CA (US)

(21) Appl. No.: 18/205,668

(22) Filed: Jun. 5, 2023

Related U.S. Application Data
(60) Provisional application No. 63/349,480, filed on Jun. 6, 2022.

(51) Int. Cl. C12Q 1/6841; C12Q 1/6806

(52) U.S. Cl. CPC C12Q 1/6841; C12Q 1/6806; C12Q 2600/16

(57) ABSTRACT

This disclosure provides a technology for optimally retrieving and presenting RNA in tissue samples for analysis by in situ hybridization, simultaneously preserving morphological and antigenic features of the tissues. Formalin-fixed, paraffin-embedded (FFPE) sections of a tissue sample are dried on glass slides, and deparaffinized by incubating in successive mixtures of organic solvents. The slides are then placed in a pressure chamber, where they are uniformly heated to about 120° C. in an atmosphere of 30 psi. After cooling, the slides are prepared for in situ hybridization and other types of analysis. The RNA retrieval process of this disclosure preserves tissue morphology, antigenic epitopes, and other features. The tissue is thereby optimized for a multiomics workflow, and for higher multiplex detection of genes and proteins in the tissue.

### RELATED APPLICATION

This application claims priority to U.S. Provisional Application Ser. No. 63/349,480, filed Jun. 6, 2022. The priority application is hereby incorporated herein by reference in its entirety for all purposes.

### FIELD OF THE INVENTION

The technology disclosed and claimed below relates generally to the fields of nucleic acid biology and the analysis and characterization of tissue sections. More specifically, it provides reagents and techniques for preparing tissue samples for in situ hybridization and multiomic analysis.

### BACKGROUND OF THE INVENTION

It is of great interest to the biotechnology community and pharmaceutical industry to develop methods for visualizing and quantifying multiple analytes within a biological sample. Research scientists and clinicians use such methods to diagnose and monitor disease, validate biomarkers, and investigate treatment. Such methods include multiplex imaging of biological samples using labeled oligonucleotides and antibodies.

Fluorescence in situ hybridization (FISH) is a technique that is used for the spatial detection and quantification of nucleic acids in their cellular environment. FISH functions via the principles of nucleic acid thermodynamics, whereby complementary strands of nucleic acids readily anneal to each other under appropriate conditions to form a duplex, a process known as a hybridization. FISH methodologies have been developed to target RNA and thus visualize gene expression in cells.

Single-cell transcriptome imaging allows quantitative measurements of both the gene expression profiles of individual, spatially localized cells and the intracellular distributions of the transcripts. At the tissue level, cell-specific gene expression defines cell types and cell states, the spatial organization of which is tightly coupled to both the development and function of normal tissues and to the pathogenesis and prognosis of tissue pathology from patients.

Multiplexed fluorescence in situ hybridization (mFISH) is a powerful imaging technique to determine gene expression in spatial transcriptomics (the subcellular localization of mRNA). A sample is exposed to multiple oligonucleotide probes that target RNA of interest. The probes have different labeling schemes that allow the user to distinguish different RNA species when the complementary, labeled probes are introduced to the sample. Sequential rounds of fluorescence images are acquired with exposure to excitation light of different wavelengths. For each given pixel, fluorescence intensities from the different images for the different wavelengths of excitation light form a signal sequence.

The detected sequence is then compared to a library of reference codes that associates each code with a gene. The best matching reference code is used to identify an associated gene that is expressed at that pixel in the image. Spatial transcriptomics permits visualization of expression of genes of interest spatially within cells and tissues. Depending on the technology used, mFISH is capable of profiling hundreds to thousands of RNA molecules in single cells. Spatially resolved RNA profiling of individual cells can be done for a range of gene transcripts with high accuracy and high detection efficiency.

The value of the analysis depends on sensitivity and accuracy of the hybridization reaction, which may be improved by implementing the materials and methods put forth in the description that follows.

### SUMMARY

This disclosure provides a technology for optimally presenting RNA in tissue samples for analysis by in situ hybridization, simultaneously preserving morphological and other features of the tissues. Formalin-fixed, paraffin-embedded (FFPE) sections of a tissue sample are dried on glass slides, and deparaffinized by incubating in successive mixtures of organic solvents. The slides are then placed in a pressure chamber, where they are uniformly heated to about 120° C. in an atmosphere of 30 psi. After cooling, the slides are prepared by chosen protocols for in situ hybridization and other types of analysis. The RNA retrieval process of this disclosure preserves tissue morphology, antigenic epitopes, and other features. The tissue is thereby optimized for a multiomics workflow, and for higher multiplex detection of genes and proteins in the tissue.

Various aspects, embodiments, features, and characteristics of the disclosure are described in the sections that follow, the accompanying drawings, and the appended claims.

### BRIEF DESCRIPTION OF THE DRAWINGS

FIG. 1 provides a workflow whereby this technology may be used to determine protein expression at a subcellular level. A formalin-fixed, paraffin-embedded (FFPE) tissue sample is exchanged into aqueous buffer. It is then probed with a nucleic acid detection system for localizing different mRNA molecules, and/or with an antibody-based detection system for localizing different gene expression products. The reagents generate different fluorescent signals for each mRNA and each protein in the sample, which are separately detected and localized. The data obtained thereby can be used for quantitative analysis and to generate visual images.

FIG. 2 shows quantitative results obtained from FFPE sections of mouse brain. From top to bottom, the graphs show signal intensity, background intensity, and spot quality for the dyes listed on the X axis. Median spot quality was approximately 1.55.

FIG. 3 is a Pearson correlation between detected copy numbers of mRNA species and their gene abundance from reference resources calculated using a log scale.

FIG. 4A shows single-cell resolution achieved in FFPE mouse brain samples probed for RNA expressed from the Cnr1, Mobp, and Gad2 genes. Experimental results are shown the two left panels, compared on the right side with an image from the Allen Mouse Brain Atlas.

FIG. 4B is a graph obtained from simultaneous multiomics visualization of RNA transcripts from three selected genes and two selected protein antigens.

FIG. 5 is a Pearson correlation of six independent experiments (with various RNA quality of RIN score and DV200%) conducted using the same workflow established for FFPE mouse brain samples. The data demonstrate that there is no batch effect or technical variability, since the correlation was close to 1.

FIGS. 6A and 6B is an intensity graph showing staining intensity of a variety of different RNA transcripts observed in a samples of mouse brain. The different transcripts are assigned to different clusters according to their known or predicted frequency of expression in different cell types.

FIG. 7 shows the level of gene expression within individual cells in the section. The left panel shows all transcripts detected in the different regions of the section labeled in accordance to their morphological characterization. The middle panel shows expression of gene clusters corresponding to neurons. The right panel shows expression of gene clusters corresponding to other cells.

FIG. 8 is a uniform manifold approximation and projection (UMAP) of 11,680 cells in the tissue section, in accordance with cell type.

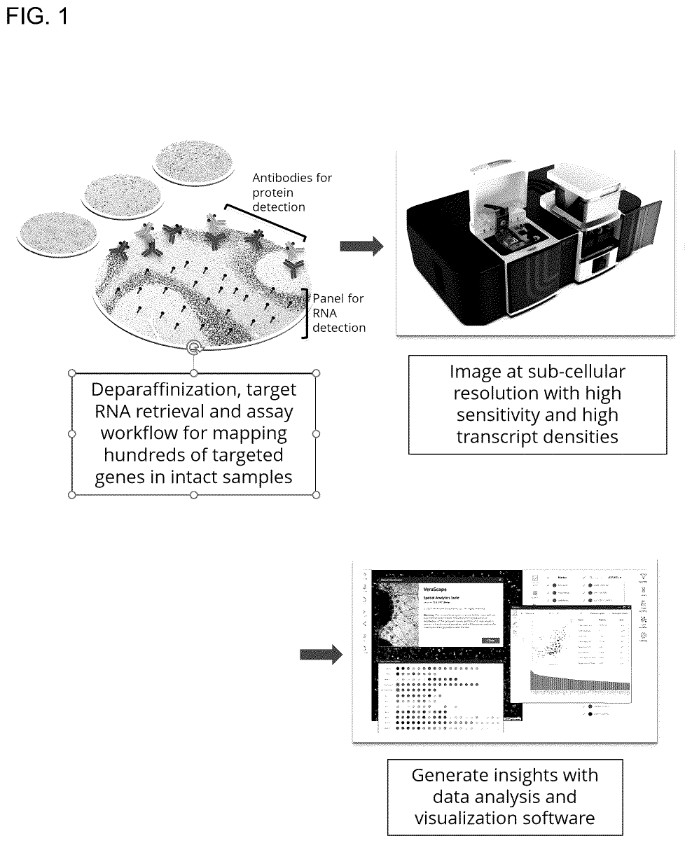

### DETAILED DESCRIPTION

This disclosure provides an optimized and standardized workflow for processing various FFPE tissue samples before analysis by in situ hybridization, optionally in combination with other types of analysis, such as immunohistochemistry. The RNA retrieval process is rapid compared with previously used technologies (it can be done in about 45 min). Preparing tissue sections in this way results in quality enhancement and improved throughput performance. It also expands the biological sample types and quality suitable for analysis by in situ hybridization.

Advantages of the RNA Retrieval Process of this Disclosure

Current technologies for sample preparation are unsatisfactory in a number of respects. There are performance quality and throughput issues, and reproducibility issues between samples from the same batch. RNA in FFPE samples prepared in the usual manner can be degraded or fragmented, leading to poor signal, and reduced probe accessibility and detection. Preparation of FFPE samples can also lead to high levels of autofluorescence. Standard preparation techniques often need to be adjusted or reoptimized for different types of tissue samples, making it especially difficult to achieve standardization.

Prior to development of the RNA retrieval process of this disclosure, FFPE samples have been prepared, for example, by enzymatic or chemical treatment. This may compromise not only the RNA in the tissue, but also morphological features and epitopes needed for immunohistochemistry. Heat is sometimes used, for example, in a microwave, an autoclave, or heated water bath. Besides potentially compromising the tissue sample, these techniques cause unevenness in the quality of the sample and the analysis, both within and between different tissue sections.

The RNA retrieval process of this disclosure is the first time pressure and heat have been combined to prepare sections for in situ hybridization. The multiple benefits of this process include the following:
The process does not require usage of enzymatic digestion methods, which can destroy morphology and protein epitopes in the sample;
The process can be used to prepare samples for multiomics analysis, for higher plex detection of genes and proteins;
Rapid processing time (both heating and cooling), which helps preserve properties of the tissue and improves workflow;
The process works for a multitude of tissues between and within different species;
The process provides uniform heating and processing within each sample;
The process works for samples with low RNA integrity;
The process eliminates the need for expensive or hazardous heating equipment; and
The process enables the processing of multiple samples simultaneously or sequentially under identical conditions.

### Overview of the Technology

In general terms, the RNA retrieval process for preparing a tissue sample for in situ hybridization is done by making formalin-fixed, paraffin-embedded (FFPE) sections of the tissue sample, and drying them on glass slides or other suitable medium.

The tissue sections are deparaffinized by incubating in a succession of solvents: typically starting with an organic solvent consisting of or containing xylene, and then with several solutions of ethanol diluted with increasing amounts of purified water, a few minutes at a time. After deparaffinizing, the sample is stored in a biologically neutral solvent, such as phosphate buffered saline (PBS). Just before heating, the storage solution is exchanged with a processing buffer. The slides are then placed in a high pressure heating chamber. Preferably, slides processed concurrently are each placed in a separate sample chamber within the apparatus.

By way of illustration, the apparatus used for the heating and pressurizing is constructed and operated in the manner of a bench-top model for thermally processing slides of the formalin-fixed, paraffin embedded tis-sues prior to immunostaining. The apparatus is designed for substantially identical processing of up to six samples concurrently, and a large number of samples in sequence. When activated, the device heats the chambers and their contents to a target temperature, at a pressure that is sufficient to achieve the target temperature. Sensors control the heating profile for the temperature and pressure to be reached at a certain pace over a certain time. After the sample is incubated at the target temperature for a desired time, the chambers are rapidly cooled, and the slides with the processed sections are retrieved.

More generally, the slides with the dried deparaffinized tissue sections are uniformly heated at a target temperature sufficient to retrieve RNA contained in the tissue to an extent desired by the user. The temperature is at least 100° C., and may be at least 110° C., 120° C., 125° C., 130° C., or 150° C., or between 100° C. and 150° C., or between 120° C. and 130° C. The pressure is sufficient to achieve the desired temperature without boiling of the sample: for example, at least 15 pounds per square inch (psi) for 100° C., at least 23 psi for 110° C.; at least 30 psi for 120° C., at least 35 psi for 125° C., at least 40 psi for 140° C., and at least 70 psi for 150° C.

The processing buffer is typically buffered at a pH of 8 (i.e., above 7.0 and below 9.0) using a buffering compound that has a pKa within one pH unit of 8: for example, POPSO, tricine, hydrazine, glycylglycine, Trizma® base (Tris), EPPS, HEPPS, BICINE, HEPBS, and TAPS. Ethylenediaminetetraacetic acid (EDTA) can be quite effective, for example, at a concentration of about 2 to 10 or 3 to 5 g per liter, adjusted to pH 8. Depending on how the technology is implemented, the processing solution also optionally contains a biocompatible amount of a salt such as NaCl, one or more preservatives, one or more enzymes or digestive agents, and/or one or more crosslinking agents, typically with the proviso that such additives do not substantially compromise morphological features of the tissue, its RNA content, and its antigenic content, and do not substantially inhibit retrieval of the RNA content or the antigenic content.

It is often advantageous to decrease the amount of processing buffer in each chamber so that it only partially fills the high pressure chamber during the heating and pressurizing: for example, only 50 mL in the 70 mL chambers of the EMS Retriever. The samples are uniformly heated in the processing chambers for a period sufficient to retrieve or reveal a desired proportion of the RNA and the antigenic components of the tissue: typically 10 to 60 min, 15 to 30 min, or at least 20 min. The samples are then allowed to cool so as not to impair the quality of the processed tissue, typically attaining a temperature near room temperature or below 30° C. or 37° C. within 10 to 30 min, or about 20 min.

Depending on its implementation, the RNA retrieval process of this disclosure may preserve and reveal a number of transcripts per cell area that is at least 70%, 90%, or more of the number of transcripts per cell area determined from a cryopreserved section of a sample obtained from the same tissue, or at least 0.3 or 0.5 transcripts per square micron. Depending on its implementation, the RNA retrieval process of this disclosure may preserve and reveal a number of tissue specific antigens per cell area that is at least 70%, 90%, or more of the number of antigen per cell area determined from a cryopreserved section of a sample obtained from the same tissue. Depending on its implementation, the RNA retrieval process of this disclosure may preserve and reveal at least 70%, 90%, or more of the morphological features that are visible in a cryopreserved section of a sample obtained from the same tissue.

Without limiting the practice of this technology, the mechanism by which the RNA in the tissue is retrieved is believed to include improving access of hybridizing reagents, for example, by improving proximity of the RNA to the surface, retracting surrounding components of the tissue that sterically inhibit access of the detecting reagents, or stabilizing presentation of the RNA, for example, by crosslinking or solidifying the matrix underneath.

After retrieving RNA in a tissue sample according to this procedure, the sample can be used for in situ hybridization analysis: for example, using multiplexed DNA probes complementary to RNA contained in the tissue. The sample may also be analyzed by immunohistochemistry using antibodies or other binding agents (such as antibody fragments or lectins) specific for tissue-specific antigens in the tissue. Methods of multiplex in situ hybridization and multiomics analysis are described further in the sections below.

### Detailed Protocol

By way of illustration, the technology described herein may be implemented as follows:
1 Formalin-fixed, paraffin-embedded (FFPE) tissue is sliced into sections of 5 μm thickness onto glass slices, and dried 37° C. overnight;
2. Deparaffinize the tissue as follows (conducted at RT):
   Incubate tissue sections in xylene for 3 min each, twice;
   Incubate tissue sections in 100% ethanol for 2 min, twice;
   Incubate tissue sections in 95% ethanol for 2 min, twice;
   Incubate tissue sections in 70% ethanol for 2 min, twice;
   Dip tissue sections in purified (distilled, MiliQ® or DEPC-treated) water for sec;
3. Leave in 1×PBS until needed;
4. Conduct RNA retrieval of each coverslip in a separate holding tank or partition of the pressurizing apparatus;
5. Cool the samples in the machine for 20 min;
6. Rinse samples in 1×PBS.

The RNA retrieval is done, for example, using a Retriever 2100 model pressurized heater obtained from Electron Microscopy Sciences, Hatfield PA, United States. Deparaffinized sections on slides are placed into the Processing (Tissue Slide) chambers. The device can accommodate simultaneously from 1 to 6 chambers at once. The individual chambers are each filled with 50 mL (rather than 70 mL) of processing buffer. The device is then filled and closed. The start button is pressed, whereupon the samples are incubated at high pressure and temperature (>120° C.) for 20 min.

The RNA retrieval is done in 50 mL of 1× of “Buffer B” (pH 8), also obtained from Electron Microscopy Sciences. Buffers having similar characteristics can be made, for example, using ethylenediaminetetraacetic acid (EDTA) at a concentration of 0.37 g per liter, adjusted to pH 8.0 using NaOH.

The deparaffinized and retrieved tissue sample is then prepared for in situ hybridization and/or immunohistochemistry according to what is required for the analytical technology selected. For example, tissue may be prepared for in situ hybridization as follows:
7 Incubate the FFPE sample with Sample Prep Wash Buffer (formamide, 2×SSC-saline solution) for 2 h at 47° C.;
8. Incubate the sample with the hybridization mix in the oven at 47° C. overnight;
9. After overnight incubation, remove the sample from the oven, place it in a petri dish and rinse the sample with the Sample Prep Wash Buffer; Aspirate the remaining buffer in the petri dish and incubate the sample with fresh
10. Sample Prep Wash Buffer for 15-30 min at 47° C.;
11. Repeat the preceding step;
12. Aspirate the remaining buffer in the petri dish and add Cycling Buffer (2×SSC-saline solution) at RT for rinsing;
13. Repeat the preceding step;
14. After the last rinsing step, aspirate the remaining buffer and add fresh Cycling Buffer to the petri dish. Seal the petri dish with parafilm and store at 4° C.;
15. Proceed with imaging using the spatial analyzer when user is ready.

### In Situ Hybridization Images Obtained Following the RNA Retrieval Process

As a demonstration of this technology, samples of mouse brain, mouse liver, mouse spleen, and human brain prepared using the RNA retrieval process have been analyzed by in situ hybridization using the standard procedure in a PISCES™ system apparatus (described below).

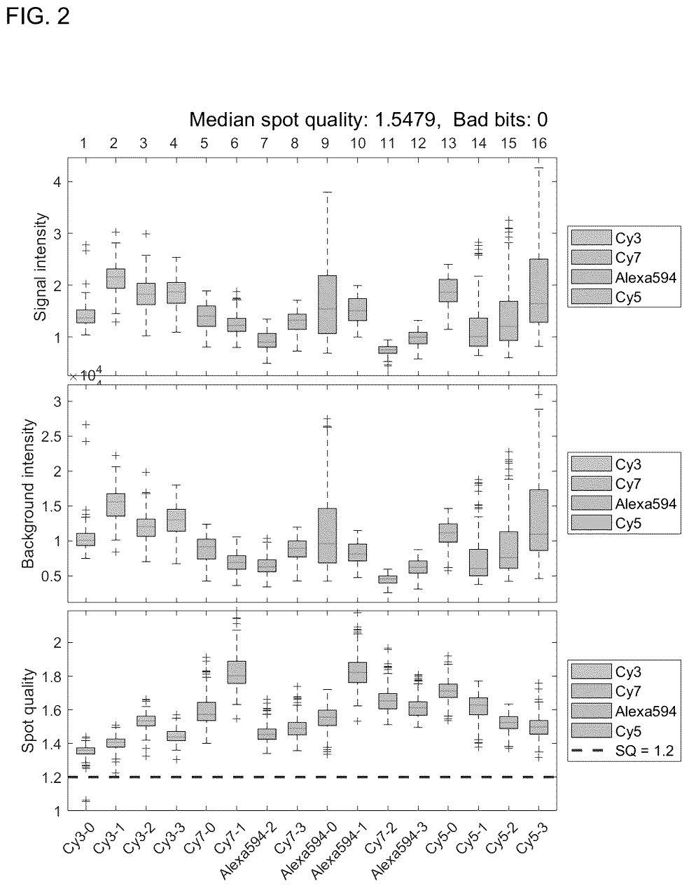

FIG. 2 shows results obtained from FFPE sections of mouse brain. From top to bottom, the graphs show signal intensity, background intensity, and spot quality for the dyes listed on the X axis. Signal intensity is the average intensity of spots of each mRNA species for that wavelength being imaged. Background intensity is the average intensity of background of the tissue samples for that wavelength being imaged. Spot quality is the normalized signal intensity with respective to background intensity generated. Median spot quality is the medial value of normalized signal intensities with respect to background intensity being determined for the different wavelengths for all the images being acquired.

The spot quality and signal intensity were consistently above one, with low background. Median spot quality was approximately 1.55.

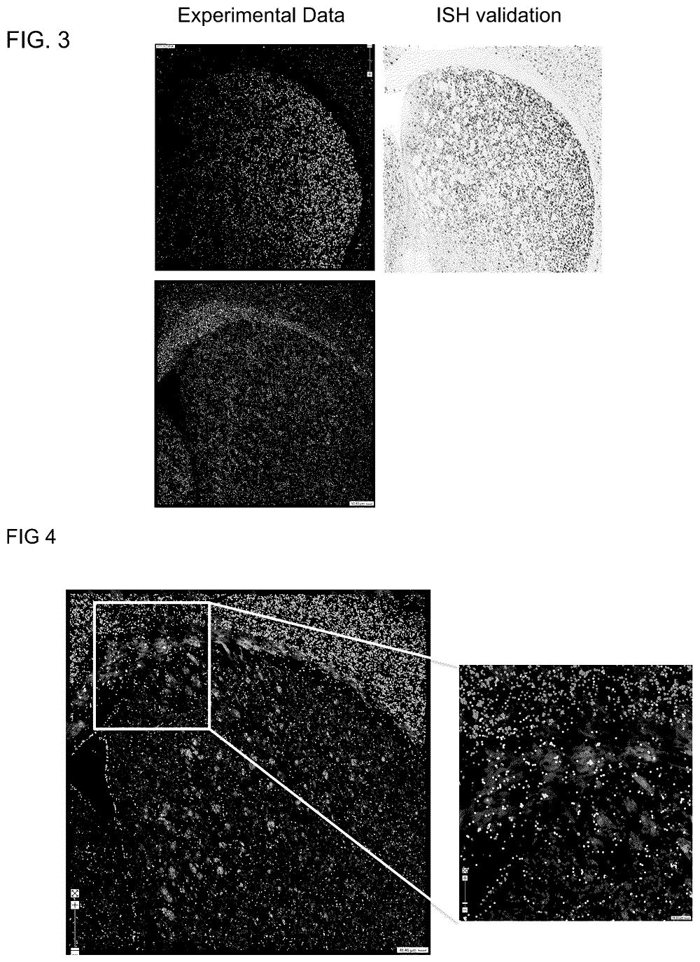

FIG. 3 shows a Pearson correlation between detected copy numbers of mRNA species and their gene abundance from reference resources (for example, from bulk RNA sequencing) calculated at log scale. Comparable results were obtained for the mouse brain FFPE and cryosection in all metrics stated in the table. There were no significant differences between the results obtained for these two sample types.

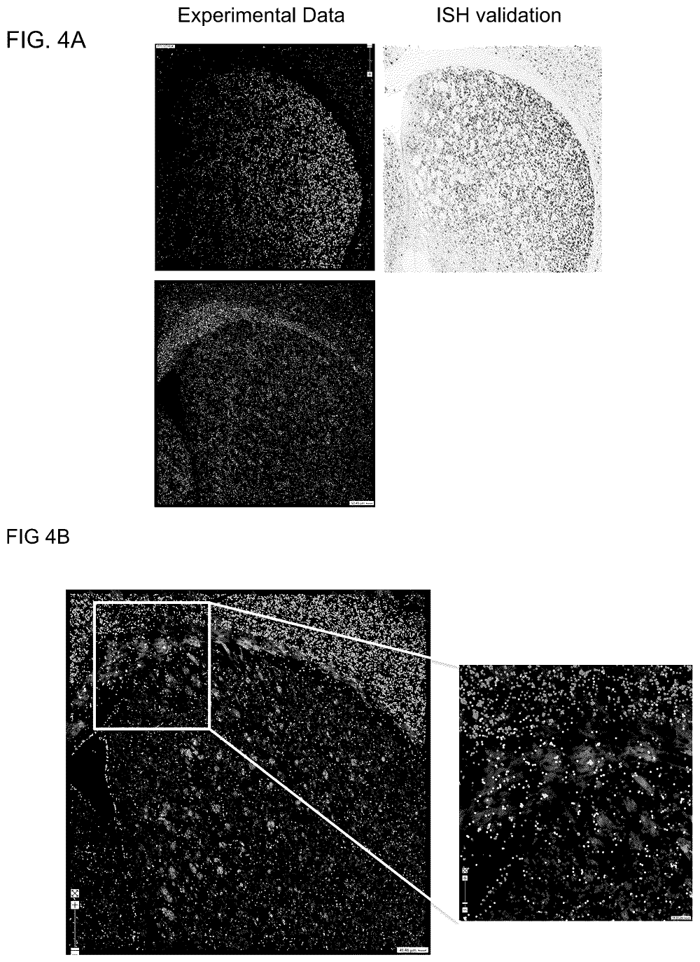

FIG. 4A shows single-cell resolution achieved in FFPE mouse brain samples probed for RNA encoding the Cnr1, Mobp, and Gad2 genes. Results in the two left panels were compared with the image shown on the right side, obtained from the Allen Mouse Brain Atlas, a full-color, high-resolution anatomic reference atlas accompanied by a systematic, hierarchically organized taxonomy of mouse brain structures.

FIG. 4B shows simultaneous multiomics visualization of RNA transcripts from three genes (Tbr 1, Rtn1, and Aqp4), and two protein antigens (Gfap and Mbp).

### Quality of Images Obtained

Samples prepared using the RNA retrieval protocol of this disclosure generated reproducible and robust in situ hybridization images, irrespective of the RNA quality in the FFPE samples.

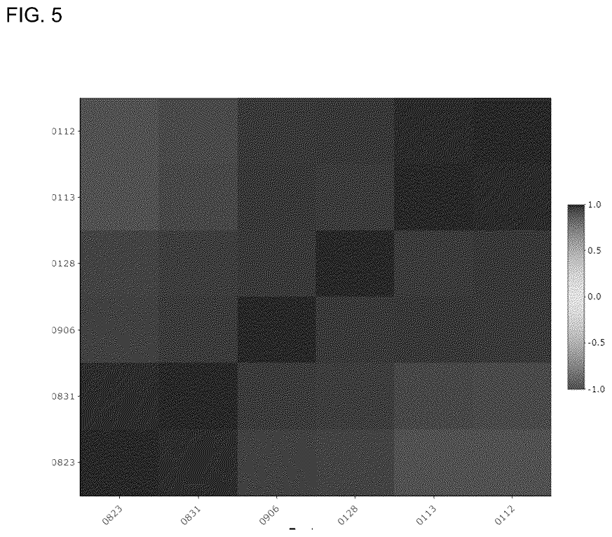

FIG. 5 shows a Pearson correlation of six independent experiments (with various RNA quality of RIN score 3.45 or 4.69, and DV200% of 38.95% or 52.8%) conducted using the same workflow established for FFPE mouse brain samples. The correlation is for results obtained from the average expression of all genes (transcripts per area) in the respective samples. “DV200%” is the percentage of fragmented RNA detected that is >200 nucleotides, which is a gauge for RNA quality for FFPE tissue samples. The higher the number, the higher the RNA quality of the sample. “RIN” measures the ratio of 18S to 28S ribosomal subunits. It is a classification of eukaryotic total RNA, based on a numbering system from 1 to 10, with 1 being the most degraded and 10 being the most intact. When RNA is degraded, there is a decrease in the 18S to 28S ribosomal band ratio and an increase in the baseline signal between the two ribosomal peaks and the lower marker.

The data in this figure demonstrate that there is no batch effect or technical variability (since the correlation was close to 1). The data also demonstrate that the FFPE workflow established in this filing is suitable to accommodate variabilities in RNA quality such that the results remain comparable or similar to one another.

### Cluster Analysis of Different Cell Types in the Same Tissue

The RNA retrieval process of this disclosure provides samples that can be used to simultaneously distinguish eleven different cell types and gene expression profiles in FFPE mouse brain samples.

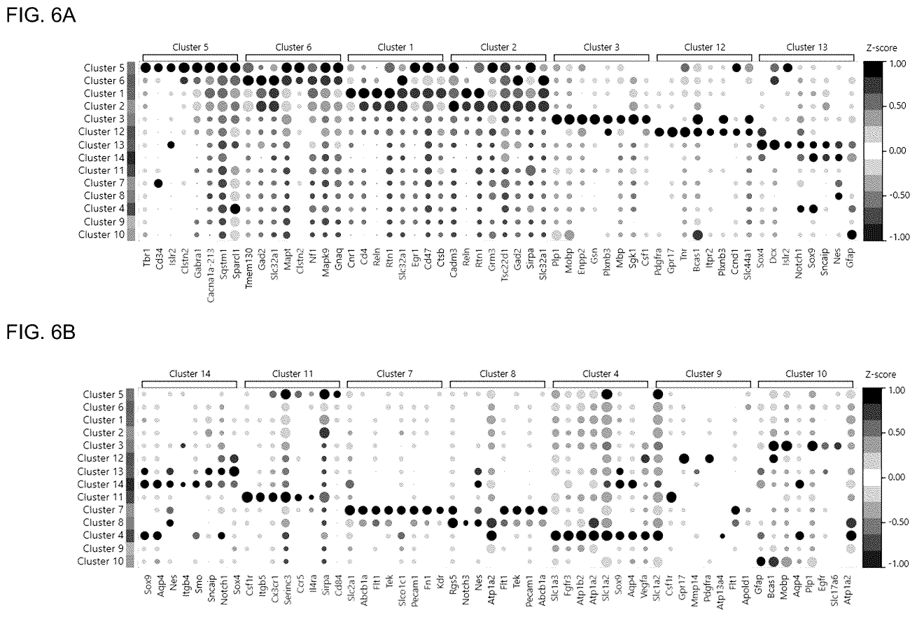

FIGS. 6A and 6B show the staining intensity of a variety of different RNA transcripts observed in the samples of mouse brain. The genes tested are listed on the X axis; the diameter of the spot is proportional to the count of each transcript in the tissue. The different transcripts are assigned to different clusters according to their known or predicted frequency of expression in different cell types. Clusters 1 and 2 belong to the inhibitory neuron group.

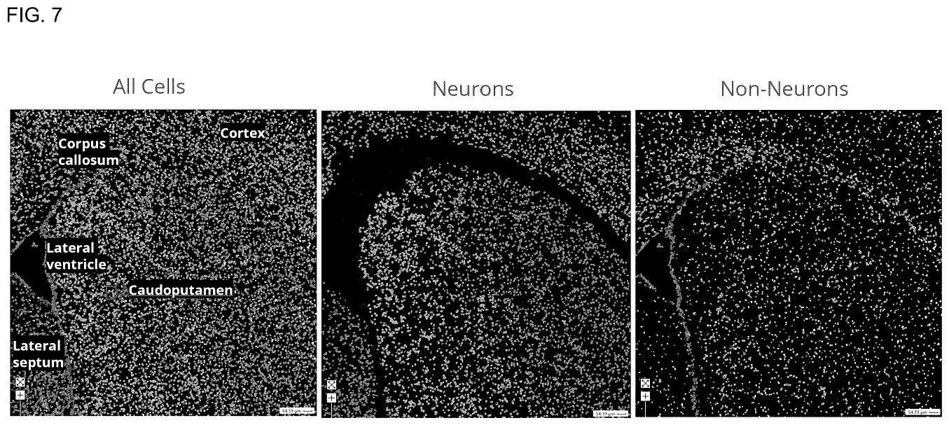

FIG. 7 shows the expression profile of individual cells in the section. The left panel shows all transcripts detected in the different regions of the section labeled in accordance to their morphological characterization. The middle panel shows expression of gene clusters corresponding to neurons. The right panel shows expression of gene clusters corresponding to other cells.

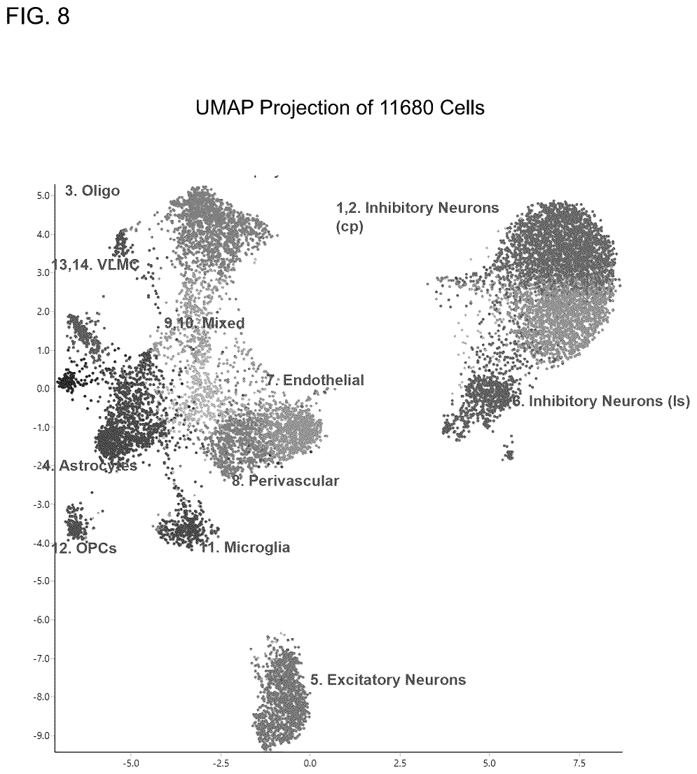

FIG. 8 is a Uniform Manifold Approximation and Projection (UMAP) of 11,680 cells in the section. The spatial localization of these clusters was validated according to the literature.

### Effectiveness on Different Types of Tissue

The tissue preparation process of this disclosure can be used to retrieve RNA in a variety of different tissues.

TABLE 1 compares data obtained from different tissues and preparations.

The correlation data shown comes from a Pearson correlation between detected copy numbers of mRNA species and their gene abundance from reference resources (for example, from bulk RNA sequencing) calculated at log scale. Normalized noise % is the amount of noise detected in tissue sample in relation to signal detected, expressed in percent. Transcripts per cell area is the total number of RNA transcripts detected in tissue sample in relation to cell area. Median spot quality is the median of normalized signal intensities with respective to background intensity being determined for the different wavelengths for all the images being acquired.

TABLE 1
Summary of metrics for different tissue samples

| | mouse | mouse | mouse | mouse | human |
|---|---|---|---|---|---|
| | brain | brain | liver | spleen | brain |
| | (cryosections) | (FFPE) | (FFPE) | (FFPE) | (FFPE) |
| correlation | 0.82 | 0.84 | 0.69 | 0.41 | 0.47** |
| normalized noise % | 6.70% | 7.93% | 11.3% | 9.94% | 7.88% |
| transcripts per cell | 0.616 | 0.599 | 0.244 | 0.039* | N/A |
| area (/μm²) | | | | | |
| median SQ | 1.51 | 1.54 | 1.42 | 1.19 | 1.37 |

*Slight dip in transcripts per cell area is normal as the spleen cells are relatively smaller, so fewer genes are detected per cell area
**Slight dip in correlation values could be attributed by patient-patient differences from bulk RNA sequencing obtained from Databank online.

Cryosections are considered to be the “gold standard” for in situ hybridization, because the tissue is fresh frozen and not chemically or enzymatically treated before contacting with the hybridization probes. The data in TABLE 1 show that comparable results can be obtained for the mouse brain FFPE and cryosection in all metrics stated in the table. There are no significant differences between the results obtained for these two sample types.

Optimal results were also obtained from other sample types (mouse liver, spleen and human brain) using the same RNA retrieval process. This shows that the RNA retrieval process is robust: it does not need to be adjusted or reoptimized between different types of tissue.

### General Technology for RNA In Situ Hybridization

The RNA retrieval process of this disclosure can be used to optimize FFPE samples for a range of different technologies for characterizing or mapping RNA in a tissue sample.

Fluorescence in situ hybridization (FISH) is a molecular cytogenetic technique in which fluorescent oligonucleotide probes hybridize to nucleic acid sequences in a tissue sample to detect and localize specific RNA targets (mRNA, lncRNA, miRNA) in tissue samples and cells. Multiplexed fluorescence in situ hybridization (mFISH) is a technique that uses a range of probes that each bind specifically to different nucleic acid targets having different sequences, with a sequential or simultaneous labeling strategy that separately identifies mRNA species having different sequences. Single-molecule fluorescent in situ hybridization (smFISH) implements short (50 bp) oligonucleotide probes conjugated with five fluorophores to obtain quantitative information about expression of certain genes in the cell. RNAscope employs probes of the specific Z-shaped design to simultaneously amplify hybridization signals and suppress background noise.

Single-molecule RNA detection at depth by hybridization chain reaction (smHCR) is an advanced seqFISH technique in which a set of short DNA probes attach to a defined subsequence of the target, followed by fluorophore-labeled DNA HCR hairpins that penetrate the sample and assemble into fluorescent amplification polymers attached to the initiating probes. Cyclic-ouroboros smFISH (osmFISH) visualizes transcripts in the manner of smFISH, and an image is acquired before the probe is stripped and the sample is reprobed. Multiplexed error-robust fluorescence in situ hybridization (MERFISH) is a single-cell transcriptome imaging method that encodes RNA target molecules with error-robust binary barcodes. The readout sequences are detected using fluorophore-labelled secondary probe, and the fluorescence signal is extinguished via photobleaching before subsequent rounds of imaging. DNA microscopy generates cDNA in fixed tissue, following which randomized nucleotides are used to tag and amplify target cDNAs in situ, thereby generating unique labels for each molecule. SeqFISH Plus resolves optical issues related to spatial crowding using a primary probe anneals to targeted mRNA, followed by readout probes that bind to flanking regions in a way that can be captured as an image and collapsed into a super-resolved image.

### General Technology for Multiomic Analysis

Multiomics is the use of multiple detection methods to detect different types of macromolecules in a tissue sample or cell preparation.

For example, any of the in situ hybridization methods referred to in the last section can be combined with cell-by-cell genomic analysis=. This is useful, for example, to obtain information regarding copy-number variation, which is not revealed by in situ hybridization alone. Alternatively or in addition, the user may implement technology to query the epigenome.

Of particular interest is the concurrent detection of both mRNA and tissue-specific process. Any of the in situ hybridization technology put forth in the previous section can be combined on the same or serial tissue sections using immunohistochemical techniques.

Devices for Carrying Out mFISH Analysis of Tissue Samples

Veranome Biosystems LLC, an Applied Materials company, has developed the PISCES™ system, an apparatus and computerized control system for conducting mFISH. Incorporated herein by reference in its entirety as part of this disclosure is US 2021/0181111 A1. The system comprises the following components:
a flow cell to contain a sample to be exposed to fluorescent probes in a reagent;
a valve to control flow from one of a plurality of reagent sources the flow cell;
a pump to cause fluid flow through the flow cell;
a fluorescence microscope including a variable frequency excitation light source and a camera positioned to receive fluorescently emitted light from the sample;
an actuator to cause relative vertical motion between the flow cell and the fluorescence microscope;
a motor to cause to cause relative lateral motion between the flow cell and the fluorescence microscope; and
a control system.

The control system operates the components of the apparatus by performing the following actions as nested loops:
cause the valve to sequentially couple the flow cell to a plurality of different reagent sources to expose the sample to a plurality of different reagents,
for each reagent of the plurality of different reagents, cause the motor to sequentially position the fluorescence microscope relative to sample at a plurality of different fields of view,
for each field of view of the plurality of different fields of view, cause the variable frequency excitation light source to sequentially emit a plurality of different wavelengths,
for each wavelength of the plurality of different wavelengths, cause the actuator to sequentially position the fluorescence microscope relative to sample at a plurality of different vertical heights, and
for each vertical height of the plurality of different vertical heights, obtain an image at the respective vertical height covering the respective field of view of the sample having respective fluorescent probes of the respective regent as excited by the respective wavelength.

### Practice

The technology provided in this disclosure and its use are described within a hypothetical understanding of general principles of nucleic acid chemistry and tissue analysis. These discussions are provided for the edification and interest of the reader, and are not intended to limit the practice of the claimed embodiments. All of the products and methods claimed in this application may be used for any suitable purpose without restriction, unless otherwise indicated or required.

While embodiments have been described with reference to the specific examples and illustrations, changes can be made and equivalents can be substituted to adapt the technology to a particular context or intended use as a matter of routine development and optimization and within the purview of one of ordinary skill in the art, thereby achieving benefits of the embodiments without departing from the scope of what is claimed and their equivalents.

What is claimed is:

1. An RNA retrieval process for preparing a tissue sample for in situ hybridization of RNA contained therein, the process comprising:
obtaining formalin-fixed, paraffin-embedded (FFPE) sections of the tissue sample dried on glass slides;
deparaffinizing the FFPE sections by incubating the glass slides in a succession of solvents;
exchanging the solvents in which the slides have been deparaffinized with an aqueous processing buffer;
placing the slides in the processing buffer in a high pressure chamber;
uniformly heating and pressurizing the slides in the chamber;
cooling the slides;
exchanging the aqueous buffer with fresh solvent; and
preparing the slides for in situ hybridization of RNA contained therein.

2. The process of claim 1, wherein the heating and pressurizing are done in a chamber to at least 120° C. in an atmosphere of at least 30 pounds per square inch (psi).

3. The process of claim 1, wherein the processing buffer has a pH of 8.

4. The process of claim 1, wherein the processing buffer only partially fills the high pressure chamber during the heating and pressurizing.

5. The process of claim 1, wherein the slides are heated and pressurized in the chamber for a period of 10 to 60 min.

6. The process of claim 1, wherein the slides are cooled after the heating and pressurizing to room temperature within 30 min.

7. The process of claim 1, which preserves and reveals a number of transcripts per cell area that is at least 90% of the number of transcripts per cell area determined from a cryopreserved section of a sample obtained from the same tissue or tissue type.

8. The process of claim 1, which preserves and reveals at least 0.5 transcripts per square micron.

9. The process of claim 1, which preserves and reveals a number of tissue specific antigens per cell area that is at least 90% of the number of antigen per cell area determined from a cryopreserved section of a sample obtained from the same tissue.

10. The process of claim 1, which accurately preserves and reveals at least 90% of the morphological features that are visible in a cryopreserved section of a sample obtained from the same tissue.

11. The process of claim 1, further comprising analyzing the prepared slides by in situ hybridization using multiplexed DNA probes complementary to RNA contained in the tissue.

12. The process of claim 1, further comprising analyzing the prepared slides by immunohistochemistry using antibodies or other binding agents specific for tissue-specific antigens in the tissue.

13. A method of multiplex in situ hybridization to determine and identify RNA transcripts expressed in a tissue sample, the method comprising:
processing the tissue to retrieve RNA contained in the tissue according to claim 1; and
conducting in situ hybridization by contacting the processed tissue with multiplexed DNA probes complementary to RNA contained in the tissue.

14. A method of multiomic analysis of a tissue sample to determine and identify RNA transcripts and tissue specific antigens contained therein, the method comprising:
processing the tissue to retrieve RNA contained in the tissue according to claim 1;
conducting in situ hybridization by contacting the processed tissue with multiplexed DNA probes complementary to RNA contained in the tissue; and
conducting immunohistochemistry by contacting the processed tissue with antibodies or other binding agents that bind specifically to antigens in the tissue.
